(19) United States
(12) Patent Application Publication
(10) Pub. No.: US 2021/0346642 A1
(43) Pub. Date: Nov. 11, 2021

(54) SYSTEMS AND METHODS FOR SLEEP INDUCEMENT THROUGH CORE BODY TEMPERATURE COOLING

(71) Applicant: Somnibliss, LLC, New Hope, PA (US)

(72) Inventors: Michael Reed, New York, NY (US); Margaret Reed, New York, NY (US)

(21) Appl. No.: 17/243,720

(22) Filed: Apr. 29, 2021

Related U.S. Application Data
(63) Continuation of application No. PCT/US2019/059029, filed on Oct. 31, 2019.
(60) Provisional application No. 62/754,263, filed on Nov. 1, 2018.

(51) Int. Cl. A61M 21/02; A61F 7/00; A61F 7/02

(52) U.S. Cl. CPC A61M 21/02; A61F 7/007; A61F 7/02; A61M 2205/3606; A61F 2007/0075; A61F 2007/0292; A61M 2021/0066; A61F 2007/0036; A61F 2007/0047; A61M 2205/3673; A61M 2210/083; A61M 2210/086; A61M 2205/3646; A61M 2209/088

(57) ABSTRACT

The present invention provides systems and methods inducing sleep through core body temperature cooling. The system comprises a primary heat sink in thermal contact with the wearer's palms and/or soles of feet causing cooling of the Arteriovenous Anastomoses (AVA's). The resulting controlled lowering of the core body temperature induces sleep.

### RELATED APPLICATIONS

This application is a continuation of International Application No. PCT/US2019/059029, which designated the United States and was filed on Oct. 31, 2019, published in English, which claims priority to U.S. Provisional Application No. 62/754,263, filed on Nov. 1, 2018. The entire teachings of the above applications are incorporated herein by reference.

### FIELD OF THE INVENTION

The present disclosure relates to systems and methods for assisting users in falling asleep through core body temperature cooling. More particularly, to sleep assisting devices and methods comprising a cooling device which lower the body's core temperature via the blood vessels found in the palms of the hands and the soles of the feet.

### BACKGROUND OF THE INVENTION

Most experts in the science of sleep agree that body temperature is a key part of what regulates the circadian rhythm that determines when the body is ready to go to sleep. When falling asleep, the body's temperature lowers one to two degrees Fahrenheit. This is why taking a warm bath helps one fall asleep; it raises the body's temperature slightly, and the process of it lowering mimics the body's natural sleep temperature pattern.

It is well known that blood vessels in the palms of the hands and soles of the feet are key in regulating the body's core temperature, not only the skin temperature. The prior art discloses various devices which cool the blood vessels in the palms. Embodiments include specialized gloves and wraps, or more simple configurations which merely strap across the palm. However, these devices and their uses are specifically directed towards athletic performance improvement and muscle recovery. Adaptation of these devices for use as a sleep aid by altering circadian rhythms has not been contemplated.

For example, in U.S. Pat. No. 9,370,207 by Crowder, claim 1 is very specific in requiring use “during a session of physical exertion”. Col 9, Ln 29-33. The method further requires “manually withdrawing air from the interior liner . . . wherein manually withdrawing air includes withdrawing air by using the mouth of the person wearing the glove”. Id Col 9, Ln 43-48. Clearly, use of the device and/or method is not compatible with inducing sleep.

As another example, claim 1 of U.S. Pat. No. 8,641,745 by Warner et al., describes a method for extracting heat from a human body. However, like Crowder, step d of the method requires “engaging in physical exertion activity”.

Therefore, a system or method to assist a person in falling asleep that is capable of altering circadian rhythms through controlled cooling of blood vessels in the palms of a human hand or soles of the feet is highly desirable.

### SUMMARY OF THE INVENTION

The present invention provides a system and method for inducing sleep through core body temperature cooling. The system comprises a primary heat sink, a thermal charging stand in thermal communication with the primary heat sink, a cooling module in thermal communication with the thermal charging stand, a base controller in electrical communication with the cooling module, and a power supply for the base controller and cooling module. The primary heat sink is applied to the wearers palms or feet, causing cooling of the wearer's Arteriovenous Anastomoses (AVA's). The resulting controlled lowering of the core body temperature mimics natural circadian rhythms thereby inducing sleep.

### BRIEF DESCRIPTION OF THE DRAWINGS

FIG. 1—System overview depicting a first half and a second half of a primary heat sink, a thermal charging stand, a cooling module, and a base controller.

FIG. 2—Cutaway view of primary heat sink showing machined channels to contain phase change material.

FIG. 3—Cutaway view of primary heat sink showing optional fins.

### DETAILED DESCRIPTION OF THE INVENTION

The terms “a”, “an” and “the” as used herein are defined to mean “one or more” and include the plural unless the context is inappropriate.

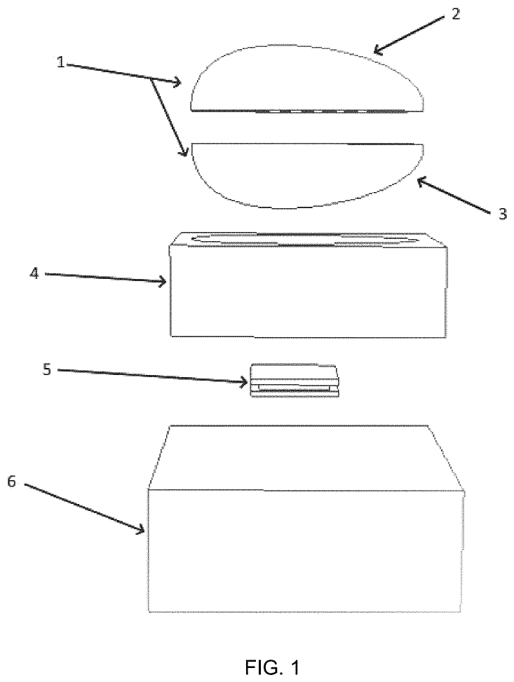

The present invention provides a system and method for inducing sleep through cooling the core temperature of a human subject. As shown in FIG. 1, the system comprises a primary heat sink 1, a thermal charging stand 4 in thermal communication with the primary heat sink 1, a cooling module 5 in thermal communication with the thermal charging stand 4, a base controller 6 in electrical communication with the cooling module 5, and a power supply in electrical communication with the base controller 6. The base controller 6 is configured to adjust the cooling module 5 to establish and maintain a predetermined temperature of the thermal charging stand 4 and to disable the cooling module 5 when a predetermined period has passed. The primary heat sink 1 is placed in thermal contact with at least one of the palms or soles of a human subject so as to cause cooling of the Arteriovenous Anastomoses (AVA's), thereby mimicking the natural pattern of temperature-lowering during the onset of sleep.

Prior to use, the primary heat sink 1 is cooled to a desired temperature. Preferably, the desired temperature will result in the human subject's body temperature cooling within a range of between one to four degrees Celsius as compared to the human subject's normal body temperature. When the primary heat sink 1 is applied to the palms or feet of the human subject, the core body temperature cooling should occur over a period of time that induces sleep, preferably about 30 minutes. During this time, the temperature of the primary heat sink 1 should gradually return to ambient.

In a preferred embodiment, the cooling module is a thermoelectric cooler comprising a hot side and a cold side. The cold side is in thermal communication with the thermal charging stand. To optimize the performance of the thermoelectric cooler, a secondary heat sink may be placed in thermal communication with the hot side of the thermoelectric cooler.

In another embodiment, the primary heat sink 1 can be constructed of a light weight, thermally conductive material, such as aluminum or ceramic. The primary heat sink 1 may further be configured to contain, internally, a phase change material 7 (PCM), such as glycerin or paraffin. Although many PCMs are available and could be substituted for paraffin or glycerin, there are practical considerations during selection. For example, it is desirable to select a material that has a melting temperature within the anticipated operating temperature range such that the PCM is a solid before use and melts during use, using the heat absorbed from the subject as the heat source for the PCM's phase transition at an overall substantially constant temperature. Furthermore, chemical stability at storage temperatures and operating temperatures is desirable. It is also desirable that the material's volume does not change substantially during cycling, which may result in an increase or decrease in pressure, thereby complicating containment. Materials that degrade after numerous freeze/melt cycles are also desirably avoided. Preferably, the primary heat sink 1 is configured into a geometry that is comfortably worn during rest or sleep. For example, an egg shape (or ovoid) sized at the approximate contour of a palm can be desired. A small adult hand, for example, can have an egg shape, or ovoid, approximately 5 cm in length and 3 cm in the width, as measured along the widest cross section. A medium adult hand, for example, can have an egg shape, or ovoid, approximately 6 cm in length and 3.5 cm in the width, as measured along the widest cross section. A large adult hand, for example, can have an egg shape, or ovoid, approximately 7 cm in length and 4 cm in the width, as measured along the widest cross section. The primary heat sink 1 will be generally convex such that it can be comfortably received by the palm with good to excellent physical contact along the surface. Similarly, the primary heat sink 1 can have the approximate contour of the bottom of a foot, e.g., along the subject's arch. It can be desirable to manufacture the primary heat sink 1 or layer the primary heat sink 1 with a thermally conductive and malleable material that will comfortably conform to the subject's personal contour. Viscoelastic polyurethane, such as the product called Memory Foam, can be used. The primary heat sink 1 is preferably received by and rests within the thermal charging stand.

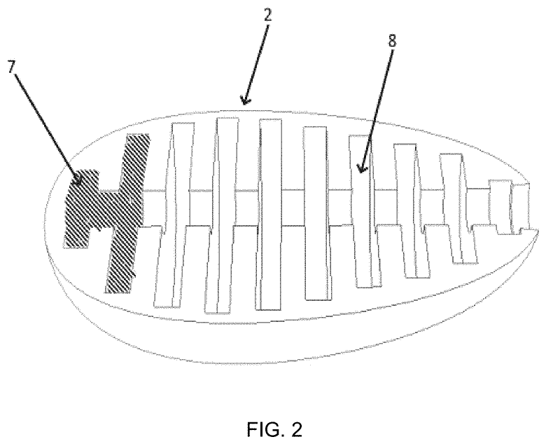

The primary heat sink 1 may be optionally be configured to comprise a first half 2 and a second half 3. Preferably, as shown in FIG. 2, at least one channel 8 is machined into at least one of the first half 2 and second half 3. The at least one channel 8 can be designed to provide optimal dispersion geometry for the phase change material 7. For example, the at least one channel 8 shown in the FIG. 2 allows the phase change material 7 to distribute more evenly throughout the first half 2 and the second half 3, and also to approach the exterior surface of the primary heat sink 1 for more effective thermal transfer. The at least one channel 8 need not be symmetrical between the first half 2 and the second half 3. The primary heat sink 1 can also be optionally configured with symmetric or asymmetric pieces, across any axis or plane, more than two pieces or one piece.

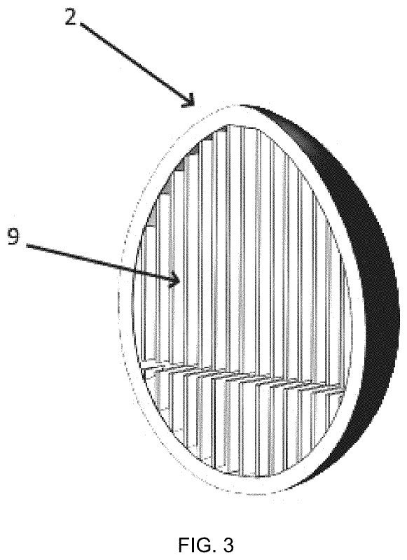

In yet another embodiment as shown in FIG. 3, the first half 2 and second half 3 comprise a plurality of fins 9 and the phase change material 7 is deposited within the spaces between the plurality of fins. Depending on the phase change material 7 selected, this geometry may allow for better thermal transfer as the phase change material 7 is more evenly dispersed and closer to the surface of the primary heat sink 1.

During the method of use, the primary heat sink 1 is in physical contact with the palms or feet of a human body. The primary heat sink 1 can be secured by a garment, such as a strap, glove, mitten or sock. The strap can be affixed to the primary heat sink 1 by, for example, holes within the primary heat sink's 1 body or it can simply be secured or attached to the exterior with glue, velcro or the like. The primary heat sink 1 can be disposed within a glove, mitten or sock and can be in direct or indirect contact with the user's palm or foot. For example, the primary heat sink 1 can be disposed within a pocket integrated into the glove, mitten or sock. A material, such as cloth, may also be disposed between the palms or feet of the subject and the primary heat sink.

The present invention also provides a method for cooling the core temperature of a human body to induce sleep, the method comprising placing a primary heat sink 1 as described herein to one or more palms or feet of a human subject prior to the human subject retiring for sleep, wherein the primary heat sink 1 is at a temperature effective to controllably cause a drop in the subject's core body temperature within a range of between one to four degrees Celsius, preferably between one and two degrees, over a period of time that induces sleep, e.g., about 30 minutes. Preferably, the primary heat sink 1 is configured to return to ambient temperature after the period of time.

The invention also provides a method for recharging the primary heat sink 1 comprising the steps of providing a thermal charging stand 4 in thermal communication with the cooling module 5; providing a primary heat sink 1 in thermal communication with the thermal charging stand 4; providing a base controller 6 in electrical communication with the cooling module 5 and configured to adjust the cooling module 5 to establish and maintain a temperature of the thermal charging stand 4 and to disable the cooling module 5 when a desired period has passed; and providing a power supply in electrical communication with the base controller 6. The primary heat sink 1 can then be placed in thermal contact with the palms or feet of a human subject so as to cause cooling of the Arteriovenous Anastomoses (AVA's).

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for cooling the core temperature of a human subject to induce sleep, the system comprising:
a. a primary heat sink;
b. a thermal charging stand in thermal communication with the primary heat sink;
c. a cooling module in thermal communication with the thermal charging stand;
d. a base controller in electrical communication with the cooling module and the base controller configured to:
i. adjust the cooling module to establish and maintain a predetermined temperature of the thermal charging stand;
ii. disable the cooling module when a predetermined period has passed;
e. a power supply in electrical communication with the base controller;
and wherein the primary heat sink can be placed in thermal contact with at least one of the palms or soles of a human subject so as to cause cooling of the Arteriovenous Anastomoses (AVA's).

2. The system of claim 1 wherein the cooling module is a thermoelectric cooler comprising a hot side and a cold side and wherein the cold side is in thermal communication with the thermal charging stand.

3. The system of claim 2 further comprising a secondary heat sink in thermal communication with the hot side of the thermoelectric cooler.

4. The system of claim 1 further comprising a phase change material disposed within the primary heat sink.

5. The system of claim 4 wherein the phase change material comprises paraffin or glycerin.

6. The system of claim 1 wherein the primary heat sink is ovoid in shape.

7. The system of claim 1 wherein the primary heat sink comprises a first half and a second half.

8. The system of claim 7 wherein the first half and the second half comprise internal channels.

9. The system of claim 1 further comprising a garment and wherein the garment affixes the primary heat sink to the palms or feet of the human subject.

10. The system of claim 9 wherein the garment comprises a strap, a glove, a mitten or a sock.

11. The system of claim 10 further comprising a material that separates the palms or feet of the human subject and the primary heat sink.

12. A method for cooling the core temperature of a human body to induce sleep, the method comprising the steps of:
a. cooling a primary heat sink to a desired temperature;
b. applying the primary heat sink to the palms or feet of a human subject prior to the subject retiring for sleep;
and wherein the desired temperature of step a is effective to controllably cause a drop in the human subject's core body temperature with a range of between one and four degrees Celsius and over a period of time that induces sleep.

13. The method of claim 12 wherein the primary heat sink returns to ambient temperature after the period of time.

14. A method for recharging a primary heat sink, the method comprising the steps of:
a. providing a thermal charging stand in thermal communication with a cooling module;
b. providing a primary heat sink in thermal communication with the thermal charging stand;
c. providing a base controller in electrical communication with the cooling module and configured to:
i. adjust the cooling module to establish and maintain a predetermined temperature of the thermal charging stand;
ii. disable the cooling module when a predetermined period has passed;
d. providing a power supply in electrical communication with the base controller and wherein the primary heat sink may then be placed in thermal contact with the palms or feet of a human subject so as to cause cooling of the Arteriovenous Anastomoses (AVA's).
